(19) United States
(12) Patent Application Publication
(10) Pub. No.: US 2007/0270929 A1
(43) Pub. Date: Nov. 22, 2007

(54) Apparatus and Method for Treatment of a Mammal

(76) Inventor: Hendrik Jacobus De Villiers, Pretoria (ZA)

Correspondence Address: BIRCH STEWART KOLASCH & BIRCH, PO BOX 747, FALLS CHURCH, VA, 22040-0747, US

(21) Appl. No.: 11/662,259

(22) PCT Filed: Sep. 9, 2005
(86) PCT No.: PCT/IB05/02675

§ 371 Date: Mar. 9, 2007

(30) Foreign Application Priority Data
Sep. 10, 2004 (ZA) 2004/7278

(51) Int. Cl. A61N 1/02

(52) U.S. Cl. 607/149

(57) ABSTRACT

The invention comprises an apparatus for use in the treatment of a mammalian patient to at least partly stimulate regeneration of cells in the patient's body. The apparatus includes means to generate a combined output signal and means to connect the combined output signal to a patient. The combined output signal is generated by summing at least two frequency modulated square wave signals having different unmodulated frequencies and which have been modulated by the same amount of frequency modulation.

### FIELD OF THE INVENTION

This invention relates to an apparatus for the treatment of a mammalian patient to stimulate regeneration of tissue cells and a method for using the apparatus in a mammal.

### BACKGROUND TO THE INVENTION

Mammalians undergo deterioration of cells in their bodies. When the mammal is still young and growing such deterioration may be offset by normal growth, but with aging the ability of the body to regenerate diminishes. Consequently, aging is associated with cell deterioration, for example the loss of muscle tissue with aging.

In some instances cell deterioration is associated with a specific illness, in which case specific dysfunctional cells may be recognized as a cause for such illness. An example is diabetes in which case dysfunctional beta cells reduce the pancreas' ability to manufacture insulin. This leaves excess sugar in the mammal's blood which causes specific diabetes related problems.

There have been numerous attempts to revitalize a mammal's ability to regenerate cells to delay the onset of aging or to treat specific illnesses. These include the taking of supplements, undergoing specific treatments and the like. None of these has been very successful.

### OBJECT OF THE INVENTION

It is an object of this invention to provide an apparatus and a method of using the apparatus that at least partly overcomes the mentioned problems.

### SUMMARY OF THE INVENTION

In accordance with this invention there is provided an apparatus for use in the treatment of a mammalian patient to at least partly stimulate regeneration of cells in the patient's body, the apparatus including means to generate a combined output signal and means to connect the combined output signal to a patient; the combined output signal being generated by summing at least two frequency modulated square wave signals having different unmodulated frequencies and which have been modulated by the same amount of frequency modulation.

There is also provided for the apparatus to include means to generate the unmodulated square wave signals, for the unmodulated frequency of the first square wave signal to be greater than the unmodulated frequency of the second square wave signal by a ratio of between about 133:1 and about 1.2:1, preferably by a ratio of between about 65:1 and about 1.2:1, further preferably by a ratio of about 13.2:1.

There is further provided for the apparatus to include means to generate an unmodulated first square wave signal having a frequency between 100 kHz and 1000 kHz, preferably between about 100 kHz and 650 kHz, further preferably about 6211 kHz; and means to generate an unmodulated second square wave signal having a frequency between 7.5 kHz and 85 kHz, preferably between about 10 kHz and 85 kHz, further preferably about 47 kHz.

There is still further provided for the apparatus to include means to modulate the first and second square wave signals, preferably by means of a modulating signal generator which generates a modulating square wave signal with a frequency of between 300 Hz and 350 Hz, preferably about 321 Hz.

According to a further feature of the invention there is provided for the apparatus to include means to generate a single output signal comprising an unmodulated square wave signal having a frequency of between 12 Hz and 50 Hz, preferably a frequency of about 12 Hz; and for the apparatus to include means to connect the single output signal to a patient, preferably through the connecting means of the combined output signal.

There is also provided for the square wave signals to have amplitudes which range between 5V and 15V.

There is further provided for the connecting means to comprise at least one contact pad preferably having a diameter of at least 60 mm, further preferably about 80 mm.

There is still further provided for the apparatus to include timing means configured to alternately generate the combined output signal and single output signal for limited periods of time, preferably less than about 40 minutes, further preferably for about 20 minutes, still further preferably for about 10 minutes for each of the combined output signal and single output signals respectively.

There is also provided for the timing means to be configured to generate the single and combined output signals in succession and for the single output signal to be generated before the combined output signal.

According to a still further feature of the invention there is provided a method of treating diabetes in a mammalian patient by connecting an apparatus as defined above to the patient and generating at least the combined output signal through the output means to the patient for a predetermined time and to repeat the treatment at predetermined intervals for a predetermined time.

There is further provided for the method to include generating the single output signal through the output means to the patient, preferably to generate the single and combined output signals in succession and further preferably to generate the single output single before generating the combined output signal.

There is further provided for the method to include generating the single output signal for a predetermined time, preferably for less than about 40 minutes, further preferably for less than about 20 minutes and still further preferably for about 10 minutes; and for the method to include generating the combined output signal for a predetermined time, preferably for less than about 40 minutes, further preferably for less than about 20 minutes and still further preferably for about 10 minutes.

There is still further provided for the method to include repeating the treatment every 24 to 48 hours and for the treatment to be administered for at least one week, preferably for the treatment to be administered once about every 48 hours for about 2 weeks, and thereafter to be administered once about every 24 hours.

### BRIEF DESCRIPTION OF THE DRAWINGS

An embodiment of the invention will be described below by way of example only and with reference to the accompanying representations in which:

FIG. 1 is a diagrammatic representation of an apparatus used to stimulate regeneration of beta cells in the pancreas of a human patient.

### DETAILED DESCRIPTION OF THE INVENTION

An embodiment of an apparatus and method of using the apparatus is described below. The embodiment relates to the treatment of a human patient suffering from diabetes. It is believed that the treatment stimulates regeneration of beta cells in the patient, which allows the patient's pancreas to produce at least some insulin and which causes the blood sugar level of the patient to stabilize to normal levels.

Even though the embodiment relates specifically to the treatment of a human patient with diabetes, it will be appreciated that the apparatus may be used for treatment of other illnesses or conditions as well. In such instances the frequencies generated by the apparatus will be tailored to have the best possible effect on cells associated with a specific part of the patient's body and which may be in need of regeneration. Therefore, even though only the embodiment of treating a human patient with diabetes is described here, it will be appreciated that the apparatus may be used to stimulate cell regeneration in a patient generally and the frequencies may very well differ from those described here. Such frequencies and treatments also fall within the scope of this invention.

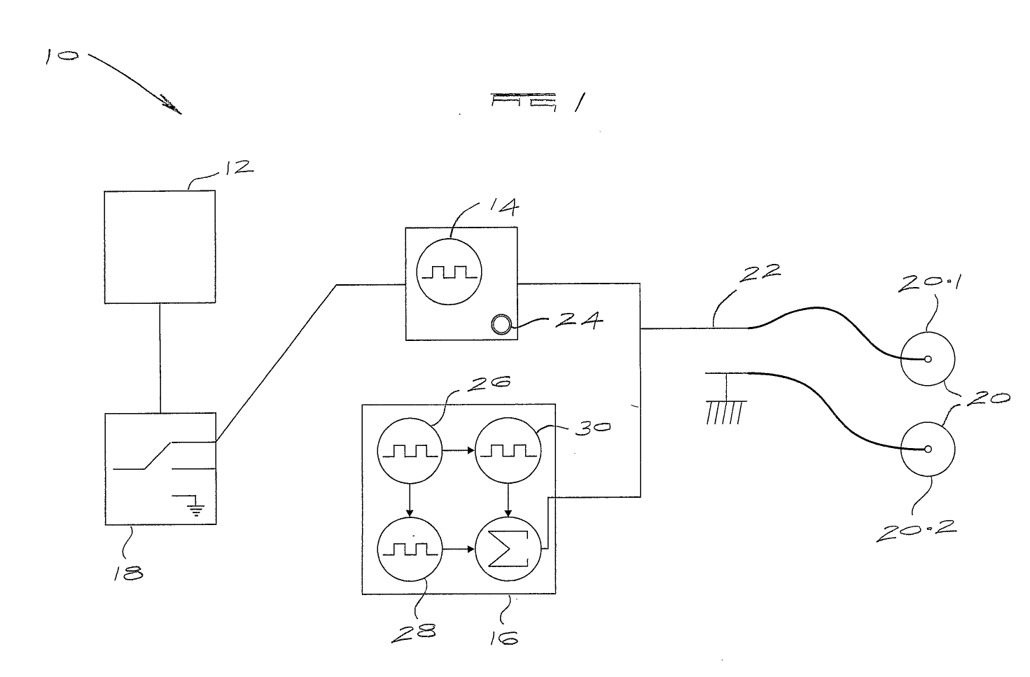

Referring to FIG. 1, the apparatus (10) has a timer (12), a 12 Hz signal generator (14), a combined output signal generator (16), a selector (18) and connectors (20).

The 12 Hz signal generator (14) generates a square wave (not shown), with a duty cycle of less than about 50% at a frequency of about 12 Hz. The 12 Hz square wave signal is applied to an output terminal (22) which in turn is connected to the body of a patient (not shown) through the connectors (20). The connectors (20) are preferably connected to the abdominal cavity or thorax of the patient. The connectors (20) consist of two standard conductive pads (20.1, 20.2) each pad having a diameter of about 80 mm. A conductive gel is applied to the contacting side of each pad and the pads are then placed spaced apart on the abdomen of the patient. The 12 Hz signal generator (14) is also fitted with a potentiometer (24) through which a patient may adjust the amplitude of the 12 Hz square wave signal so as to achieve a comfortable treatment level.

The combined output signal generator (16) has a modulating square wave generator (26) which creates a modulating square wave at a duty cycle of about 60% and a frequency of about 321 Hz. A first square wave generator (28) is configured to produce a first square wave signal at a frequency of about 621 kHz and a duty cycle of about 60%. A second square wave generator (30) is configured to produce a second square wave signal at a frequency of about 47 kHz and a duty cycle of about 60%.

The square wave signals ( ) are generated to have amplitudes between 3V and 15V.

The 321 Hz modulating square wave signal is fed into both the first and second square wave generators (28, 30) acting as a modulating signal which frequency modulates both the 621 kHz and 475 kHz signals. The output of the first square wave generator (28) is therefore a square wave having a frequency periodically varying between about 621.321 and 620.679 kHz and the output of the second square wave generator (30) accordingly creates a square wave having a frequency periodically varying between about 47.321 kHz and 46.679 kHz.

The outputs of the first and second square wave generators (28, 30) are then summed and applied to the output terminal (22) which, as described above, is then applied to the body of a patient through the connectors (20.1, 20.2).

The combination of the two frequency modulated signals created by the first and second square wave generators (28, 30) results in the creation of a wide range of harmonic frequencies.

The timer (12) is configured to provide a signal which toggles the selector (18) sequentially from a first to a second and then to a third state with a transition between states occurring only once the timer (12) has detected the lapse of a predetermined amount of time.

When the apparatus is switched on the selector (18) automatically moves to state one in which the 12 Hz signal generator (14) is activated. After spending 10 minutes in state one the timer (10) actuates the selector (18) to move to state two. When the selector (18) is in state two, the combined output signal generator (16) is activated. After spending another 10 minutes in state two, the timer (12) again actuates the selector (18) to move to state three where the apparatus (10) switches off. The apparatus (10) is also provided with a reset button (not shown) which, when pressed, causes the selector (18) to move back to state one where the treatment cycle restarts.

It is believed that the application of the signal created by the combined output signal generator (16) to the body of a patient has definite medicinal advantages. In particular if the combined output signal is created using the specific base frequencies as described above and more particularly if the ratio between the base frequencies of the second and third square wave generators (28, 30) are as above, it is believed that application of the signal to the body of a patient is specifically advantageous as a treatment for diabetes.

It will be appreciated that this is only one embodiment of an apparatus according to the invention. Even though it is possible to produce the required output signals by means of electric circuitry different from that described here such different embodiments still fall within the scope of this invention.

It should also be noted that the duty cycle of 60% used in the example is not critical to the success of the treatment. Duty cycles varying between 10% and 60% have been tested and have not to affect the results significantly. It is also envisaged that duty cycles of higher than 60% may be used with success in this treatment.

1. An apparatus for use in the treatment of a mammalian patient to at least partly stimulate regeneration of cells in the patient's body, the apparatus including means to generate a combined output signal and means to connect the combined output signal to a patient; the combined output signal being generated by summing at least two frequency modulated square wave signals having different unmodulated frequencies and which have been modulated by the same amount of frequency modulation.

2. An apparatus as claimed in claim 1 which includes means to generate the unmodulated square wave signals.

3. An apparatus as claimed in claim 1 or claim 2 in which the unmodulated frequency of the first square wave signal is greater than the unmodulated frequency of the second square wave signal by a ratio of between about 133:1 and about 1.2:1.

4. An apparatus as claimed in claim 1 or claim 2 in which the unmodulated frequency of the first square wave signal is greater than the unmodulated frequency of the second square wave signal by a ratio of between about 65:1 and about 1.2:1

5. An apparatus as claimed in claim 1 or claim 2 in which the unmodulated frequency of the first square wave signal is greater than the unmodulated frequency of the second square wave signal by a ratio of about 13.2:1.

6. An apparatus as claimed in any one of claims 1 to 5 which includes means to generate an unmodulated first square wave signal having a frequency between 100 kHz and 1000 kHz, and means to generate an unmodulated second square wave signal having a frequency between 7.5 kHz and 85 kHz.

7. An apparatus as claimed in any one of claims 1 to 5 which includes means to generate an unmodulated first square wave signal having a frequency between 100 kHz and 650 kHz, and means to generate an unmodulated second square wave signal having a frequency between 10 kHz and 85 kHz.

8. An apparatus as claimed in any one of claims 1 to 5 which includes means to generate an unmodulated first square wave signal having a frequency of about 621 kHz and means to generate an unmodulated second square wave signal having a frequency about 47 kHz.

9. An apparatus as claimed in any one of claims 1 to 8 which includes a modulating signal generator which generates a modulating square wave signal with a frequency of between 300 Hz and 350 Hz.

10. An apparatus as claimed in claim 9 in which the modulating signal generator generates a modulating square wave signal with a frequency of about 321 Hz.

11. An apparatus as claimed in any one of claims 1 to 10 which includes means to generate a single output signal comprising an unmodulated square wave signal having a frequency of between 12 Hz and 50 Hz.

12. An apparatus as claimed in claim 11 in which the single output signal has a frequency of about 12 Hz.

13. An apparatus as claimed in claim 11 or claim 12 which includes means to connect the single output signal to a patient.

14. An apparatus as claimed in claim 13 in which the means to connect the single output signal to a patient comprises the connecting means of the combined output signal.

15. An apparatus as claimed in any one of claims 1 to 14 in which the square wave signals have amplitudes which range between 5V and 15V.

16. An apparatus as claimed in any one of the previous claims in which the connecting means comprises at least one contact pad.

17. An apparatus as claimed in claim 16 in which the contact pad has a diameter of at least 60 mm.

18. An apparatus as claimed in claim 16 in which the contact pad has a diameter of at least preferably having a diameter of about 80 mm.

19. An apparatus as claimed in any one of the previous claims which includes timing means configured to alternately generate the combined output signal and single output signal for limited periods of time.

20. An apparatus as claimed in claim 19 in which the timing means is configured to alternately generate the combined output signal and single output signal for less than about 40 minutes for each of the combined output signal and single output signals respectively.

21. An apparatus as claimed in claim 19 in which the timing means is configured to alternately generate the combined output signal and single output signal for less than about 20 minutes for each of the combined output signal and single output signals respectively.

22. An apparatus as claimed in claim 19 in which the timing means is configured to alternately generate the combined output signal and single output signal for about 10 minutes for each of the combined output signal and single output signals respectively.

23. An apparatus as claimed in any one of claims 19 to 22 in which the timing means is configured to generate the single and combined output signals in succession and the single output signal is generated before the combined output signal.

24. A method of treating diabetes in a mammalian patient which includes connecting an apparatus as claimed in any one of claims 1 to 11 to a mammalian patient, generating the combined output signal through the output means to the patient for a predetermined time and repeating the treatment at predetermined intervals for a predetermined time.

25. A method as claimed in any one of claims 12 to 24 which includes generating the single output signal through the output means to the patient.

26. A method as claimed in claim 25 which includes generating the single and combined output signals in succession.

27. A method as claimed in claim 26 which includes generating the single output single before generating the combined output signal.

28. A method as claimed in any one of claims 25 to 27 which includes generating the single output signal and the combined output signal for a predetermined time.

29. A method as claimed in claim 28 which includes generating the single output signal and the combined output signal each for less than about 40 minutes respectively.

30. A method as claimed in claim 29 which includes generating the single output signal and the combined output signal each for less than about 20 minutes respectively.

31. A method as claimed in claim 29 which includes generating the single output signal and the combined output signal each for about 10 minutes respectively.

32. A method as claimed in any one of claims 24 to 31 which includes repeating the treatment about every 24 to 48 hours.

33. A method as claimed in claim 32 in which the method includes administering the treatment once about every 48 hours for about 2 weeks, followed by administering the treatment once about every 24 hours.

34. A method as claimed in any one of claims 24 to 33 which includes administering the treatment for at least one week.

35. An apparatus for use in the treatment of a mammalian patient substantially as herein described with reference to the example and FIG. 1.

36. A method of treating diabetes in a mammalian patient substantially as herein described with reference to the example.
